(19) United States
(12) Patent Application Publication
(10) Pub. No.: US 2009/0191250 A1
(43) Pub. Date: Jul. 30, 2009

(54) Antimicrobial Composite Material and Method for Fluid Treatment

(75) Inventors: Jan W. Gooch, Atlanta, GA (US); Arthur W. Johnston, Atlanta, GA (US); Arthur F. Johnston, Atlanta, GA (US)

Correspondence Address: SUTHERLAND ASBILL & BRENNAN LLP, 999 PEACHTREE STREET, N.E., ATLANTA, GA, 30309, US

(73) Assignee: WATER VISIONS INTERNATIONAL, INC., Atlanta, GA (US)

(21) Appl. No.: 12/020,896

(22) Filed: Jan. 28, 2008

(51) Int. Cl. A01N 25/26; A01N 47/44; C12M 1/00

(52) U.S. Cl. 424/405; 514/635; 435/306.1

(57) ABSTRACT

Composite materials with broad spectrum antimicrobial properties and methods and devices for fluid treatment utilizing said materials are provided. The antimicrobial composite materials may include combinations of activated carbon and a biguanide hydrate. A particular composition includes a mixture of carbon particles and particles of chlorhexidine hydrate, which is useful in fixed particle bed water treatment devices and methods.

### BACKGROUND OF THE INVENTION

This invention is generally in the field of antimicrobial materials, devices, and methods for treating fluids, such as water, air, and other gases or aqueous fluids, that are or may be contaminated with one or more microorganisms in need of deactivation.

There is a general need for improved devices and methods to eliminate microorganisms from fluids for various applications, including the provision of safe or potable drinking water and breathable purified air. Many different methods are currently used for the purification of fluids. Representative examples include distillation, ion-exchange, chemical adsorption, filtering, and retention. Oftentimes, a number of different techniques must be combined to provide complete purification of fluids. These techniques can be costly, energy inefficient, and require significant technical expertise. Unfortunately, many low cost purification techniques do not adequately treat or remove harmful biological contaminants, bacteria, and viruses.

The United States Environmental Protection Agency (SPA) has set forth minimum standards for acceptance of a device proposed for use as a microbiological water filter. Common coliforms, represented by the bacteria E. coli and Klebsiella terrigena, must show a minimum 6-log reduction (99.9999% of organisms removed) from an influent concentration of 1×10⁷ per 100 mL of water. Common viruses, represented by poliovirus 1 (LSc) and rotavirus (Wa or SA-11), which show a resistance to many treatment processes, must show a minimum 4-log reduction (99.99% of organisms removed), from an influent concentration of 1×10⁷ per 100 mL of water. Cysts, such as those represented by Giardia muris or Giardia lamblia, are widespread, disease-inducing, and resistant to most forms of chemical disinfection. A device claiming cyst-removal must show a minimum 3-log reduction (99.9% of cysts removed) from an influent concentration of 1×10⁶ per L or 1×10⁷ per L.

It is known to use strong oxidants, such as phenols and hypochlorites, to effectively negate the potential threat of all microorganisms in water, however, these agents must be removed from water before consumption. Known biocompatible antimicrobial agents generally destroy only select microorganisms rather than a broad spectrum of microorganisms, thereby requiring the use of multiple biocompatible antimicrobial agents to effectively negate the potential threat of all microorganisms.

One conventional biocompatible antimicrobial agent is known as chlorhexidine. Chlorhexidine is a 1,6-di(4-chlorophenyl-diguanido) hexane. The IUPAC name or chlorhexidine is N,N″Bis(4-chlorophenyl)-3,12-diimino-2,4,11,13-tetrazatetradecanediimideamide. Chlorhexidine has a high level of antibacterial activity, low mammalian toxicity, and a strong affinity for binding to skin and mucous membranes. It has been used as a topical antiseptic for application to areas such as skin, wounds, and mucous membranes. Chlorhexidine also has been used as a pharmaceutical preservative and as a disinfectant for inanimate surfaces. Chlorhexidine has been used in its salt soluble forms. However, these forms have an extremely bitter taste that must be masked in formulations intended for oral use and are water soluble and thus ineffective for the many applications that require the antimicrobial material to be substantially water insoluble.

In addition, chlorhexidine's antimicrobial activity is directed mainly toward vegetative gram-positive and gram-negative bacteria. It is ineffective against bacterial spores, except at elevated temperatures. Acid-fast bacilli are merely inhibited and not inactivated by aqueous solutions of chlorhexidine. At relatively low concentrations, chlorhexidine is bacteriostatic, while at higher concentrations, chlorhexidine is rapidly bactericidal. Chlorhexidine's fungicidal activity is subject to species variation. Although chlorhexidine and its known derivatives exhibit some antimicrobial activity, they unfortunately may not be effective against a broad spectrum of microorganism types.

Other water soluble antimicrobial chemical agents are known in the art. Representative examples of such conventional materials include soaps/detergents, surfactants, acids, alkalis, heavy metals, halogens, alcohols, phenols, oxidizing agents and alkylating agents. Most of these agents chemically alter (e.g., by an oxidation reaction etc.) the cellular structure of microbes to inactivate them. These agents may have undesirable side-effects on the affected area of contamination (skin, clothes, paint, etc.) with often deleterious side-effects (discoloration and oxidation).

Accordingly, there remains a need for an inexpensive and biocompatible antimicrobial agent that will effectively inactivate a broad spectrum of microorganisms. There is also a need for an antimicrobial material that is practical for use in a variety of fluid purification systems. Desirably, the antimicrobial material would significantly exceed the minimum EPA requirements for designation as a microbial water purifier such that it is suitable for consumer and industry point-of-use applications.

### SUMMARY OF THE INVENTION

Antimicrobial composite materials are provided, along with devices and methods of use and methods of making the composite material. In one aspect, the composite material may include particles of carbon and an antimicrobial material which comprises a compound having the formula

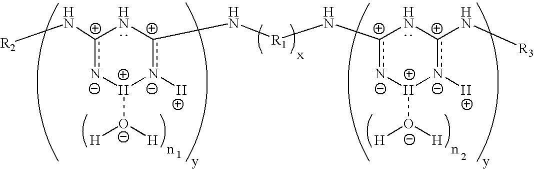

wherein R₁ comprises a straight chained, branched, or cyclic alkyl group;

wherein R₂ and R₃, independent of one another, comprise a hydrogen, halogen, hydroxyl, amino, amido, alkylamino, arylamino, alkoxy, aryloxy, nitro, acyl, alkenyl, alkynyl, cyano, sulfo, sulfato, mercapto, imino, sulfonyl, sulfenyl, sulfinyl, sulfamoyl, phosphonyl, phosphinyl, phosphoryl, phosphino, thioester, thioether, anhydride, oximno, hydrazino, carbamyl, phosphonic acid, phosphonato, or a straight, chained, branched, or cyclic alkyl, alkenyl, alkynyl, aryl, heteroaryl, or heterocyclic group;

wherein x is a number from 1 to 8 and y is a number from 1 to 4; and

wherein one or both of n₁ and n₂ is the number 1, wherein the composition has a degree of hydration greater than 0 and less than or equal to 2y.

In one embodiment, the antimicrobial composite material is in the form of a particulate mixture, wherein the antimicrobial material is in the form of particles dispersed among the particles of carbon. In one embodiment, the particles of carbon may be present in the particulate mixture in an amount from about 50% to about 85% by weight of the particulate mixture. In one embodiment, the antimicrobial material may be present in an amount from about 15% to about 50% by weight of the particulate mixture. In one embodiment, the particulate mixture may be in a porous compacted form having a volume average pore size between about 0.1 micron and about 5 microns.

In another embodiment of the antimicrobial composite material, the antimicrobial material is in the form of a coating on the particles of carbon. In one embodiment, the antimicrobial material is present in an amount from about 25% to about 60% by weight of the coated particles of carbon.

In one embodiment of the antimicrobial composite material, the carbon particles are an activated carbon, which may have a mesh size from about 40 to about 400 mesh.

In one embodiment, R₁ of the compound comprises a straight chained, branched, or cyclic alkyl group which is substituted with a moiety selected from the group consisting of hydrogen, halogen, hydroxyl, amino, amido, alkylamino, arylamino, alkoxy, aryloxy, nitro, acyl, alkenyl, alkynyl, cyano, sulfo, sulfato, mercapto, imino, sulfonyl, sulfenyl, sulfinyl, sulfamoyl, phosphonyl, phosphinyl, phosphoryl, phosphino, thioester, thioether anhydride, oximno, hydrazino, carbamyl, phosphonic acid, and phosphonato.

In various embodiments of the antimicrobial material compound, x may be 6; y may be 1; R₁ may be methylene; R₂ and R₃ may be a halo-substituted phenyl; or a combination thereof.

In a preferred embodiment, an antimicrobial composite material is provided, for treating fluids, that includes particles of an activated carbon and an antimicrobial material comprising a compound having the formula

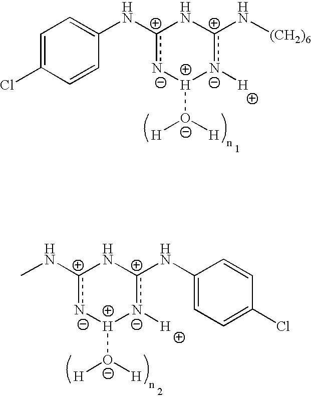

wherein one or both of n₁ and n₂ is the number 1 and the compound has a degree of hydration greater than 0 and less than 2. In one embodiment, the compound may have a degree of hydration between about 1.3 and about 1.6. The antimicrobial material may be present in an amount from about 15% to about 60% by weight of the antimicrobial composite material.

In one embodiment, the antimicrobial composite material may consist essentially of a chlorhexidine hydrate which is about 4.0% to about 5.0% water by weight.

In one embodiment of this antimicrobial composite material, the particles of activated carbon have a mesh size from about 40 to about 400 mesh. For example the particles of activated carbon may have a mesh size from 40 to 80 mesh. In another examples the particles of activated carbon may have a mesh size from 200 to 325 mesh.

In one embodiment, the antimicrobial composite material is in the form of a particulate mixture, wherein the antimicrobial material is in the form of particles dispersed among the particles of activated carbon. The particles of carbon may be present in the particulate mixture in an amount from about 50% to about 85% by weight.

In another embodiment, the antimicrobial composite material is in the form of a coating on the panicles of activated carbon.

In another aspect, a device including the antimicrobial composite material is provided for inactivating microorganisms in a fluid. The device may include a housing having at least one fluid inlet and at least one fluid outlet, and one of the foregoing antimicrobial composite materials located within the housing between the at least one fluid inlet and the at least one fluid outlet. The device may further include at least one porous polymeric layer adjacent to the antimicrobial composite material. For example, the antimicrobial composite material may be sandwiched between two porous support layers. In one embodiment, the device may further include a layer of particles of an activated carbon located between the antimicrobial composite material and the fluid outlet. In one embodiment of the device, the antimicrobial material may consist essentially of chlorhexidine hydrate which has about 4.0% to about 5.0% water by weight. In one case, the antimicrobial composite material may be in a porous compacted form having a volume average pore size between about 0.1 micron and about 5 microns.

In yet another aspect, a method is provided for inactivating microorganisms in a fluid using an antimicrobial composite material. The method may include flowing a fluid in need of treatment, such as an aqueous fluid or air, through one of the present antimicrobial composite materials in a manner effective to inactivate at least one microorganism in the fluid.

In still another aspect, a method is provided for making an antimicrobial composite material. In one embodiment, the method includes the steps of (i) providing particles of a carbon; and (ii) combining the particles of carbon with an antimicrobial material comprising a compound having the formula

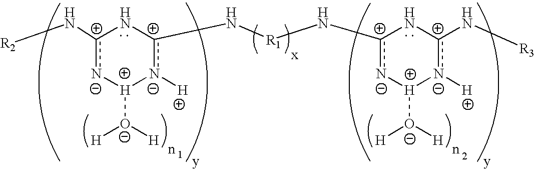

wherein R₁ comprises a straight chained, branched, or cyclic alkyl group;

wherein R₂ and R₃, independent of one another, comprise a hydrogen, halogen, hydroxyl, amino, amido, alkylamino, arylamino, alkoxy, aryloxy, nitro, acyl, alkenyl, alkynyl, cyano, sulfo, sulfato, mercapto, imino, sulfonyl, sulfenyl, sulfinyl, sulfamoyl, phosphonyl, phosphinyl, phosphoryl, phosphino, thioester, thioether, anhydride, oximno, hydrazino, carbamyl, phosphonic acid, phosphonato, or a straight, chained, branched, or cyclic alkyl, alkenyl, alkynyl, aryl, heteroaryl, or heterocyclic group;

wherein x is a number from 1 to 8 and v is a number from 1 to 4; and

wherein one or both of n₁ and n₂ is the number 1, wherein the composition has a degree of hydration greater than 0 and less than or equal to 2y.

In one embodiment of the method, the antimicrobial material is provided in the form of particles which are mixed with the particles of carbon.

In another embodiment of the method, the antimicrobial material is heated above its melting temperature, coated onto the particles of carbon, and then cooled to below its melting temperature to form a solid coating on the carbon particles.

In a certain embodiment of the method, the antimicrobial material comprises a compound having the formula

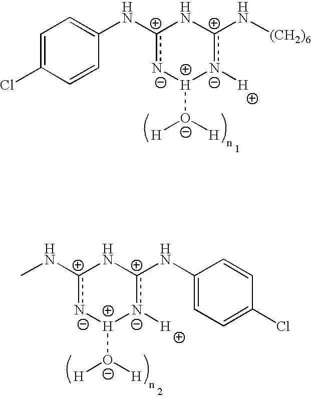

wherein one or both of n₁ and n₂ is the number 1, and wherein the compound has a degree of hydration greater than 0 and less than 2. In one embodiment of the method, the compound has a degree of hydration between about 1.3 and about 1.6.

In still another aspect, antimicrobial composite materials are provided which include particles of an inert substrate material, the particles having a volume average volume average size between about 100 microns and about 5 mm; and an antimicrobial material coated onto the particles of an inert substrate material. In one embodiment, the antimicrobial material may be a biguanide hydrate such as chlorhexidine hydrate. In various embodiments, the inert substrate material may include a ceramic, a noble metal, a polytetrafluoroethylene or a glass.

### BRIEF DESCRIPTION OF THE DRAWING

FIG. 1 is an illustration of one embodiment of a device containing an antimicrobial composite material for treating a fluid.

FIG. 2 is an illustration of another embodiment of a device containing an antimicrobial composite material for treating a fluid.

FIG. 3 is an illustration a further embodiment of a device containing an antimicrobial composite material for treating a fluid.

### DETAILED DESCRIPTION OF THE INVENTION

The biguanide hydrates described in U.S. patent application Ser. No. 11/359,422, filed Oct. 6, 2006, are effective antimicrobial materials. In one embodiment, the antimicrobial material may be provided in a particulate form within a housing through which a fluid in need of treatment flows to provide intimate contact between the fluid and the antimicrobial material. It has been found, however, that the particles of antimicrobial material may shift during fluid flow, which may create undesirable shortcut, or bypass, channels through which the fluid (and microorganisms) in need of treatment may flow, reducing the desired contact between the antimicrobial material and the microorganism to be deactivated. It has now been discovered that the positional stability of the antimicrobial particles can be improved by combining the antimicrobial biguanide hydrate material with particles of carbon material. For example, the combination may be a particulate mixture. While not wishing to be bound by any theory, it is believed that carbon particles stabilize the biguanide hydrate particles by partially filling voids between the biguanide hydrate particles.

The Composite Material

The composite material preferably includes particles of carbon combined with an antimicrobial material that includes a biguanide hydrate.

The relative amounts of biguanide hydrate and carbon present in the mixture may be modified so long as the mixture retains a useful level of antimicrobial activity. In one embodiment, the biguanide hydrate is present in an amount in the range of about 10% to about 50% by weight and the carbon is present in an amount in the range of about 50% to about 90% by weight. In another embodiment, the biguanide hydrate may be present in an amount from about 10% to about 85% by weight, and the carbon may be present in an amount from about 75% to about 90% by weight. In still another embodiment, the biguanide hydrate may be present in an amount from about 10 to about 15% by weight and the carbon may be present in an amount from about 85 to about 90% by weight.

The antimicrobial composite material may be in the form of a particulate mixture, wherein the antimicrobial material is in the form of particles dispersed among the particles of carbon. In one embodiment, the particles of carbon may be present in the particulate mixture in an amount from about 30% to about 85% (e.g., from about 50% to about 85%, from about 40 to about 75%) by weight of the particulate mixture. In one embodiment, the particulate mixture may be in a porous compacted form having a volume average pore size between about 0.1 micron and about 5 microns.

In another embodiment of the antimicrobial composite material the antimicrobial material is in the form of a coating on the particles of carbon. In various embodiments, the antimicrobial material coating may be present in an amount from about 25% to about 60% by weight of the coated particles of carbon.

The composite material may be made using essentially any suitable method for combining the biguanide hydrate and carbon particles.

In one embodiment, for example, the composite material may be made in the form of a particulate mixture, with particles of the biguanide hydrate being dispersed among the particles of carbon. That is, the mixture is a solid-solid dispersion. The mixing can be achieved using known equipment and methods. For example, a high degree of content uniformity of the composite material may be provided by using a Turbula shaker-mixer or other conventional powder blender.

In another embodiment, the antimicrobial material is coated onto the particles of carbon. In one embodiment, the biguanide hydrate is heated above its melting temperature and the fluidized biguanide hydrate is applied to the particles of carbon and then cooled to below its melting temperature to form a solid coating on the carbon particles. The steps of melting the biguanide hydrate and coating the particles of carbon can be done sequentially or simultaneously. For example, the coating process can be performed using a heated blender or mixer. The selection of suitable mixing equipment may depend in part on the relative amounts of carbon and biguanide hydrate in the composite mixture.

In a preferred embodiment of the coated particle form of the composite material, the antimicrobial material is chlorhexidine hydrate, which has a relatively low melting temperature of 90 to 95° C., which is not near the decomposition temperature for chlorhexidine. This is advantageous in that the chlorhexidine hydrate flows over the carbon particles easily without decomposing. This is an important property for coating the antimicrobial material onto a substrate. In contrast, a conventional commercial chlorhexidine would decompose before completely liquefying sufficiently to coat a substrate during a typical shearing-while-mixing operation.

In an alternative embodiment, the antimicrobial material, such as chlorhexidine hydrate, is melted and coated onto a substrate material other than carbon. In one embodiment, the biguanide hydrate, e.g., chlorhexidine hydrate, is coated onto inert particles other than activated carbon. For example the chlorhexidine hydrate, may be coated onto particles of a ceramic, glass, polytetrafluoroethylene (PTFE), or noble metal. Suitable substrate materials may be selected from those known in the art to be resistant to chemical reactions under conditions expected in a particular application of interest. In one embodiment, the particles of inert material have a volume average volume average size between about 100 microns and about 5 mm, e.g., between about 200 microns and about 2 mm, or between about 500 microns and 1 mm.

Once the composite material is made, it may be assembled or formed into essentially any form suitable for contacting a fluid in need of treatment. Such contacting arrangements and forms are known in the art. In a particular embodiment, the present antimicrobial composite material may be provided in a fixed particle bed (e.g., in a column or disk). It may be compacted in order to achieve a desired void space in the composite material. In another embodiment, the antimicrobial composite material may be extruded into various shapes for using conventional extruders and extrusion methods known in the art.

Carbon Particles

The carbon particles generally include one or more particulate forms of carbon, particularly activated carbons. Suitable carbon particles are commercially available, for example from Calgon Carbon Corporation. Activated carbon may be obtained from a variety of carbonaceous source materials (e.g. sawdust, wood, charcoal, peat, lignite, petroleum coke, bituminous coal, and coconut shells). Activated carbons generally consist of material with an exceptionally high surface area and microporosity. The particles of carbon may include a binder as known in the art.

As used herein, the term “particles” is used broadly to include various granular, powdered, or pelletized forms. Conventionally, activated carbon may be classified into broad categories based on its physical characteristics. For example, powdered activated carbon generally comprises carbons made in particular form as powders or fine granules that are less than 1.0 mm in size and having an average diameter between 0.15 and 0.25 mm. ASTM generally classifies particle sizes corresponding to an 80 mesh sieve (0.177 mm) and smaller as powdered activated carbon. Granulated activated carbon generally has larger particle sizes as compared to powdered activated carbon and may be in either granular form or extruded.

The size of the carbon particles suitable in the antimicrobial composite material may vary. It will typically be selected based on its ease of mixing with the antimicrobial material, effectiveness of stabilizing the antimicrobial material during fluid flow, and whether the material provides acceptable fluid flow rates in the particular device or method setting. For example, the carbon particles may have a volume average size between about 25 microns and about 5 mm, such as between about 50 microns and about 1 mm, between about 75 microns and about 500 microns, or between about 100 microns and about 250 microns. In one embodiment, the carbon particles may have a mesh size from about 40 to about 400 mesh. That is, at least 90% of the particles may be passed by a 40 mesh sieve and retained by a 400 mesh sieve, for example using a screen analysis as described by ASTM D 1921. In another embodiment, the carbon particles may have a mesh size from about 40 to about 395 mesh. In still another embodiment, the carbon particles may have a mesh size in the range of about 40 to about 200 mesh, from about 40 to about 140 mesh, or from about 40 to about 80 mesh.

The activated carbon may be produced using known processes, such as physical reactivation and chemical activation.

The Particles of Antimicrobial Material

The particles of antimicrobial material generally include biguanide hydrates and biguanide bases having broad spectrum antimicrobial activity, as well as tautomers of the same.

In one embodiment, the antimicrobial material is produced by reacting a chlorhexidine compound (e.g., chlorhexidine diacetate) with sodium hydroxide (or another base) to form chlorhexidine hydrate (C₂₂H₃₀N₁₀Cl₂.nH₂O). Chlorhexidine hydrate is an insoluble biguanide compound. It has an amorphous structure, which is in contrast to crystalline chlorhexidine base. Its surface energy is significantly less than many other materials which beneficially allows water or another fluid to flow through it more easily than through other materials. Chlorhexidine hydrate advantageously has a melting temperature far below its decomposition temperature, which allows it to be molded into different physical shapes without degrading the compound's chemical or structural integrity. Significantly, it has been found that chlorhexidine hydrate has broad spectrum antimicrobial activity. Chlorhexidine hydrate has been found to negate bacteria and many other kinds of microorganisms in an aqueous fluid.

The chlorhexidine hydrate disrupts the microorganisms in a principally surface-dependent manner, advantageously without depleting the supply of the chlorhexidine dihydrate. That is, chlorhexidine hydrates antimicrobial functionality is effectively catalytic. The treatment is a zero-order reaction; no chlorhexidine hydrate is consumed during treatment of a contaminated fluid. In contrast, the rate of reaction for chlorhexidine or its previously known conventional derivatives is second-order, as the reaction depends on both the concentration of chlorhexidine and the active sites of microorganisms. Conventional chlorhexidine is reacted and consumed. In contrast, chlorhexidine hydrate is particularly suitable for use in purification/treatment devices and systems due to its insolubility, amorphous structure, low surface energy, catalytic nature, and broad spectrum antimicrobial activity. In addition, it is believed that insoluble tri-guanide and tetra-guanide hydrates and bases may exhibit similar broad spectrum antimicrobial activity using the same mechanism as the biguanide hydrates and biguanide bases provided herein.

As used herein, the term “hydrate” refers to a compound formed by the addition of at least one water molecule to a host molecule. The biguanide hydrates provided herein may comprise any suitable number of water molecules (n) per biguanide molecule, wherein n may be any value between 0 and 2. For example, in a biguanide monohydrate n is 1 and in a biguanide dihydrate n is 2. The actual degree of hydration of a biguanide compound generally will be a value less than the theoretical degree of hydration and may not be an integer due to the inefficiency of the hydration reaction (i.e., the product generally will comprise a mixture of biguanide monohydrates and dihydrates). Accordingly, the actual degree of hydration may be a fraction between 0 and the theoretical degree of hydration (e.g., 0.1, 0.3, 0.5, 0.7, 1.1, 1.3, 1.4, 1.5, 1.6, or 1.7).

In one embodiment, the antimicrobial material comprises or consists essentially of a chlorhexidine hydrate which has a degree of hydration between about 1.2 and about 1.6 (e.g., 1.3 to 1.5, 1.3 to 1.4, 1.4 to 1.5).

The efficiency of the reaction and the resulting degree of hydration generally may be modified by varying different reaction conditions. Non-limiting examples of reaction conditions which may impact the resulting degree of hydration include the relative amounts or each reactant, the temperature, and the length of time the reaction is allowed to proceed.

In one embodiment, the antimicrobial material includes chlorhexidine hydrate (e.g., in particulate form) consisting essentially of a mixture of chlorhexidine monohydrate and chlorhexidine dihydrate. The chlorhexidine hydrate may be from about 4.0% to about 8.0% water by weight. In one embodiment, the chlorhexidine hydrate may be from about 4.0% to about 5.0% water by weight. In a particular embodiment, the chlorhexidine hydrate may be about 5.0% water by weight.

As used herein, the term “water insoluble” refers to substantial insolubility in aqueous fluids, particularly aqueous fluids having a pH in the range of about 3 to about 11, such as between about 4 and about 9, and particularly in the range of 6.0 to 8.0.

As used herein, the term “antimicrobial activity” refers to the property or capability of a material to inactivate microorganisms. Non-limiting examples of microorganisms include bacteria, fungi, and viruses. This “inactivation” renders the microorganism incapable of reproducing and therefore incapable of infecting other organisms and occurs by disruption of the bacteria, fungi or protozoa membrane, or by denaturization of the protein such as that which forms the protective capsid for viruses. As used herein, the term “broad spectrum antimicrobial activity” refers to the property or capability of a material to inactivate numerous different, or substantially all, types of microorganisms including bacteria (and its corresponding spores), fungi, protozoa and viruses. An antimicrobial agent that inactivates only a select group of microorganisms (e.g., either only gram positive cells or only gram negative cells) does not have broad spectrum antimicrobial activity.

In one embodiment, the antimicrobial material includes a biguanide hydrate having the chemical formula (Formula I):

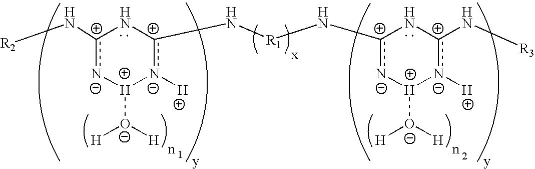

wherein R₁ comprises a straight chained, branched, or cyclic alkyl group which may be further substituted with any moieties such as hydrogen, halogen, hydroxyl, amino, amido, alkylamino, arylamino, alkoxy, aryloxy, nitro, acyl, alkenyl, alkynyl, cyano, sulfo, sulfato, mercapto, imino, sulfonyl, sulfenyl, sulfinyl, sulfamoyl, phosphonyl, phosphinyl, phosphoryl, phosphino, thioester, thioether, anhydride, oximno, hydrazino, carbamyl, phosphonic acid, phosphonato, or any other viable functional group;

wherein R₂ and R₃, independent of one another, comprise a hydrogen, halogen, hydroxyl, amino, amido, alkylamino, arylamino, alkoxy, aryloxy, nitro, acyl, alkenyl, alkynyl, cyano, sulfo, sulfato, mercapto, imino, sulfonyl, sulfenyl, sulfinyl, sulfamoyl, phosphonyl, phosphinyl, phosphoryl, phosphino, thioester, thioether, anhydride, oximno, hydrazino, carbamyl, phosphonic acid, phosphonato, or a straight chained, branched, or cyclic alkyl, alkenyl, alkynyl, aryl, heteroaryl, or heterocyclic group, which may be further substituted with any moieties such as hydrogen, halogen, hydroxyl, amino, amido, alkylamino, arylamino, alkoxy, aryloxy, nitro, acyl, alkenyl, alkynyl, cyano, sulfo, sulfato, mercapto, imino, sulfonyl, sulfenyl, sulfinyl, sulfamoyl, phosphonyl, phosphinyl, phosphoryl, phosphino, thioester thioether, anhydride, oximno, hydrazino, carbamyl, phosphonic acid, phosphonato, or any other viable functional group;

wherein n₁ and n₂, independent of each other, are numbers from 0 to 1; and

wherein x and y, independent of each other, are numbers from 1 to 3000. In certain embodiments, y is a number from 1 to 4, and x is a number from 1 to 100, from 1 to 20, from 1 to 10, or from 1 to 8. In particular embodiments, the composition has a degree of hydration greater than 0 and less than 2y.

In one embodiment, the antimicrobial material having the chemical formula I comprises a biguanide hydrate in which n₁ and n₂ are 1 having the chemical formulae

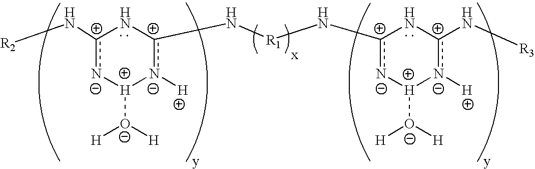

wherein R₁ comprises a straight chained, branched, or cyclic alkyl group which may be further substituted with any moieties such as hydrogen, halogen, hydroxyl, amino, amido, alkylamino, arylamino, alkoxy, aryloxy, nitro, acyl, alkenyl, alkynyl, cyano, sulfo, sulfato, mercapto, imino, sulfonyl, sulfenyl, sulfinyl, sulfamoyl, phosphonyl, phosphinyl, phosphoryl, phosphino, thioester, thioether, anhydride, oximno, hydrazino, carbamyl, phosphonic acid, phosphonato, or any other viable functional group.

wherein R₂ and R₃, independent of one another, comprise a hydrogen, halogen, hydroxyl, amino, amido, alkylamino, arylamino, alkoxy, aryloxy, nitro, acyl, alkenyl, alkynyl, cyano, sulfo, sulfato, mercapto, imino, sulfonyl, sulfenyl, sulfinyl, sulfamoyl, phosphonyl, phosphinyl, phosphoryl, phosphino, thioester, thioether, anhydride, oximno, hydrazino, carbamyl, phosphonic acid, phosphonato, or a straight chained, branched, or cyclic alkyl, alkenyl, alkynyl, aryl, heteroaryl, or heterocyclic group, which may be further substituted with any moieties such as hydrogen, halogens hydroxyl, amino, amido, alkylamino, arylamino, alkoxy, aryloxy, nitro, acyl, alkenyl, alkynyl, cyano, sulfo, sulfato, mercapto, imino, sulfonyl, sulfenyl, sulfinyl, sulfamoyl, phosphonyl, phosphinyl, phosphoryl, phosphino, thioester, thioether, anhydride, oximno, hydrazino, carbamyl, phosphonic acid, phosphonato, or any other viable functional group, and

wherein x and y, independent of each other, are numbers from 1 to 3000. In some embodiments, y is a number from 1 to 4, and x is a number from 1 to 100, from 1 to 20, from 1 to 10, or from 1 to 8. In some embodiments, the composition has a degree of hydration greater than 0 and less than 2y.

In selecting suitable or viable substitutions, the functional group desirably does not eliminate or substantially impair the broad spectrum antimicrobial activity or chemical stability of the compound. For example, R₁ generally should not be an unsaturated compound because it would prevent the transfer of electrons via double or triple bonds, disturbing the tautomerism on each side of the biguanide that is responsible for the partial charge of the guanide groups. R₁ may, however, include an isolated double or triple bond non-conjugated with other carbon atoms and with a single bond carbon atom (or more than one carbon atom) adjacent the guanide groups because the double or triple bond would not have electronic communication with the guanide groups and would not interfere with the tautomerism necessary for stabilization of the partial charges on each of the guanide groups. A further example relates to functional groups R₂ and R₃, which should be electron-withdrawing groups which are capable of assisting in the stabilization of the compound. In one particular embodiment, the biguanide hydrate of Formula I comprises chlorhexidine hydrate, having the chemical formula

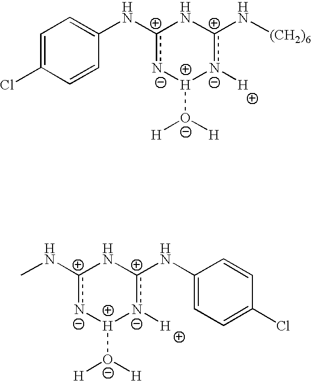

wherein R₁ is methylene, R₂ and R₃ are chloro-phenyl, n₁ is 1, n₂ is 1, x is 6, and y is 1. In a particular embodiment, the composition has a degree of hydration that is greater than 0 and less than 2.

In another embodiment of the biguanide hydrate of Formula I, R₂ and R₃, independent or one another, are electron-withdrawing groups.

In still other embodiments of the biguanide hydrate of Formula I, R₂ and R₃ are independently aryls, are independently substituted aryls, or are independently phenyls. In another embodiment of the biguanide hydrate of Formula I, R₂ and R₃ are independently substituted phenyls. The independently substituted phenyls may have ortho, para, or meta substitutions. The independently substituted phenyls may be identical to or different from one another.

In still another embodiment of the biguanide hydrate of Formula I, R₂ and R₃ are independently substituted halo phenyls. The independently substituted halo phenyls may have ortho, para, or meta substitutions. The independently substituted halo phenyls may be identical to or different from one another.

In various other examples of the biguanide hydrate of Formula I, R₂ and R₃ may independently be substituted halogens, substituted amines, substituted amides, substituted cyanos, or substituted nitros.

In another embodiment, the antimicrobial material comprises a biguanide base having the chemical formula (Formula II):

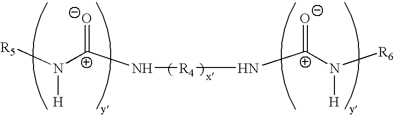

wherein R₄ comprises a straight chained, branched, or cyclic alkyl group, which may be further substituted with a hydrogen, halogen, hydroxyl, amino, amido, alkylamino, arylamino, alkoxy, aryloxy, nitro, acyl, alkenyl, alkynyl, cyano, sulfo, sulfato mercapto, imino, sulfonyl, sulfenyl, sulfinyl, sulfamoyl, phosphonyl, phosphinyl, phosphoryl, phosphino, thioester, thioether, anhydride, oximno, hydrazino, carbamyl, phosphonic acid, phosphonato, or any other viable functional group; R₅ and R₆, independent of one another, comprise a hydrogen, halogen, hydroxyl, amino, amido, alkylamino, arylamino, alkoxy, aryloxy, nitro, acyl, alkenyl, alkynyl, cyano, sulfo, sulfato, mercapto, imino, sulfonyl, sulfenyl, sulfinyl, sulfamoyl, phosphonyl, phosphinyl, phosphoryl, phosphino, thioester, thioether, anhydride, oximno, hydrazino, carbamyl phosphonic acid, phosphonato, or a straight chained, branched, or cyclic alkyl, alkenyl, alkynyl, aryl, heteroaryl, or heterocyclic group, which may be further substituted with a hydrogen, halogen, hydroxyl, amino, amido, alkylamino, arylamino, alkoxy, aryloxy, nitro, acyl, alkenyl, alkynyl, cyano, sulfo, sulfato, mercapto, imino, sulfonyl, sulfenyl, sulfinyl, sulfamoyl, phosphonyl, phosphinyl, phosphoryl, phosphino, thioester, thioether, anhydride, oximno, hydrazino, carbamyl, phosphonic acid, phosphonato, or any other viable functional group; and x′ and y′, independent of one another, are numbers from 1 to 3000. In certain embodiments, is a number from 1 to 4, and x′ is a number from 1 to 100, from 1 to 20, from 1 to 10, or from 1 to 8.

In one particular embodiment, the biguanide base of Formula II comprises a chlorhexidine base having the chemical formula

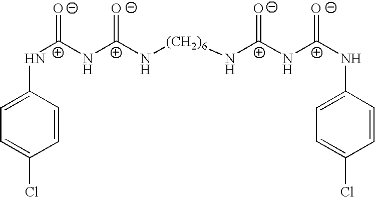

wherein R₄ is methylene, R₅ and R₆ are chloro-phenyl, x′ is 6, and NV is 2.

One skilled in the art will appreciate that the charge depicted in the biguanide base of Formula II is not a static charge on a single atom, but rather is an illustration of the net effect of a stabilized partial charge in the chemical compound. Not wishing to be bound by any theory, this charge has been well demonstrated to be the result of a combination of stabilizing tautomers.

In another embodiment of the biguanide base of Formula II, R₅ and R₆ are independently electron-withdrawing groups.

In various other embodiments of the biguanide base of Formula II, R₁ and R₆ are independently aryls, are independently substituted aryls, are independently phenyls. In one particular embodiment of the biguanide base of Formula II, R₅ and R₆ are independently substituted phenyls. The independently substituted phenyls may have ortho, para, or meta substitutions. The independently substituted phenyls may be identical to or different from one another.

In another particular embodiment of the biguanide base of Formula II, R₅ and R₆ are independently substituted halo phenyls. The independently substituted halo phenyls may have ortho, para, or meta substitutions. The independently substituted halo phenyls may be identical to or different from one another.

In various other examples of the biguanide base of Formula II, R₅ and R₆ are independently substituted halogens, substituted amines, substituted amides, substituted cyanos, or substituted nitros.

The term “alkyl”, as used herein, unless otherwise specified, refers to a saturated straight, branched, or cyclic, primary, secondary, or tertiary hydrocarbon of C1 to C20, and specifically includes methyl, ethyl, propyl, isopropyl, butyl, isobutyl, t-butyl, pentyl, isopentyl, neopentyl, hexyl, and isohexyl. The term includes both substituted and unsubstituted alkyl groups. Moieties with which the alkyl group can be substituted are selected from the group consisting of hydrogen, halogen, hydroxyl, amino, amido, alkylamino, arylamino, alkoxy, aryloxy, nitro, acyl, alkenyl, alkynyl, cyano, sulfo, sulfato, mercapto, imino, sulfonyl, sulfenyl, sulfinyl, sulfamoyl, phosphonyl, phosphinyl, phosphoryl phosphino, thioester, thioether, anhydride, oximno, hydrazino, carbamyl, phosphonic acid, phosphonato, or any other viable functional group.

The term “alkenyl”, as referred to herein, and unless otherwise specified, refers to a straight, branched, or cyclic hydrocarbon of C2 to C10 with at least one double bond. The alkenyl groups can be optionally substituted in the same manner as described above for the alkyl group and can also be optionally substituted with a substituted or unsubstituted alkyl group.

The term “alkynyl”, as used herein, and unless otherwise specified, refers to a C2 to C10 straight or branched hydrocarbon with at least one triple bond. The alkynyl groups can be optionally substituted in the same manner as described above for the alkyl groups and can also be optionally substituted with a substituted or unsubstituted alkyl group.

The term “aryl”, as used herein, and unless otherwise specified, refers to any functional group or substituent derived from an aromatic ring. Non-limiting examples include phenyl, biphenyl, and napthyl. The term includes both substituted and unsubstituted moieties. The aryl group can be substituted with one or more moieties as described above for the alkyl groups or a substituted or unsubstituted alkyl group.

The term “heteroaryl” or “heteroaromatic”, as used herein, refers to an aromatic or unsaturated cyclic moiety that includes at least one sulfur, oxygen, nitrogen, or phosphorus in the aromatic ring. Non-limiting examples are furyl, pyridyl, pyrimidyl, thienyl, isothiazolyl, imidazolyl, tetrazolyl, pyrazinyl, benofuranyl, benothiophenyl, quinolyl, isoquinolyl, benzothienyl, ixobenzofuryl, pyrazolyl, indolyl, isoindolyl benimidazolyl, purinyl, carbazolyl oxazolyl, thiazolyl, isothiazolyl, 1,2,4-thiadiazolyl, isoxazolyl, pyrolyl, quinazolinyl, pyridazinyl, pyrazinyl, cinnolyl, phthalazinyl, quinoxalinyl, xanthinyl, hypoxanthinyl, and pteridnyl. The heteroaryl or heteroaromatic group can optionally be substituted with one or moieties as described above for the alkyl group or a substituted or unsubstituted alkyl group.

The term “heterocyclic” refers to a saturated nonaromatic cyclic group which may be substituted, and wherein there is at least one heteroatom or non-carbon atom, such as oxygen, sulfur, nitrogen, or phosphorus in the ring. The heterocyclic group can be substituted in the same manner as described above for the heteroaryl group.

The term “aralkyl”, as used herein, and unless otherwise specified, refers to an aryl group as defined above linked to the molecule through an alkyl group as defined above. The term alkaryl, as used herein, and unless otherwise specified, refers to an alkyl group as defined above linked to the molecule through an aryl group as defined above. The aralkyl or alkaryl group can be optionally substituted with one or more moieties selected from the group consisting of hydroxyl, carboxy, carboxamido, carboalkoxy, acyl, amino, halo, alkylamino, alkoxy, aryloxy nitro, cyano, sulfo, sulfato, phospho, phosphato, or phosphonato.

The term “halo”, as used herein, specifically includes chloro, bromo, iodo, and fluoro.

The term “alkoxy”, as used herein, and unless otherwise specified, refers to a moiety of the structure —O-alkyl, wherein alkyl is as defined above.

The term “acyl”, as used herein, refers to a group of the formula C(O)R′, wherein R′ is an alkyl, aryl, heteroaryl, heterocyclic, alkaryl or aralkyl group, or substituted alkyl, aryl, heteroaryl, heterocyclic, aralkyl or alkaryl, wherein these groups are as defined above.

Methods of making the antimicrobial materials are described in U.S. patent application Ser. No. 11/539,422, filed Oct. 6, 2006, and in U.S. Ser. No. 12/016,550, filed Jan. 18, 2008, the latter disclosure of which is hereby incorporated by reference.

The starting materials are commercially available or may be synthesized or prepared according to methods known in the art. In one embodiment, the antimicrobial compound is made by reacting a biguanide compound (e.g., chlorhexidine or a salt of chlorhexidine) with a base, such as sodium hydroxide. The biguanide compounds have the chemical formula (Formula III):

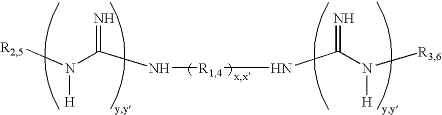

wherein R_{1,4} comprises a straight, chained, branched, or cyclic alkyl group which may be further substituted with any moieties such as hydrogen, halogen, hydroxyl, amino, amido, alkylamino, arylamino, alkoxy, aryloxy, nitro, acyl, alkenyl, alkynyl, cyano, sulfo, sulfato, mercapto, imino, sulfonyl, sulfenyl, sulfinyl, sulfamoyl, phosphonyl, phosphinyl, phosphoryl, phosphino, thioester, thioether, anhydride, oximno, hydrazino, carbamyl, phosphonic acid, phosphonato, or any other viable functional group; R_{2,5} and R_{3,6}, independent of one another, comprise a hydrogen, halogen, hydroxyl, amino, amido, alkylamino, arylamino, alkoxy, aryloxy, nitro, acyl, alkenyl, alkynyl, cyano, sulfo, sulfato, mercapto, imino, sulfonyl, sulfenyl, sulfinyl, sulfamoyl, phosphonyl, phosphinyl, phosphoryl, phosphino, thioester, thioether, anhydride, oximno, hydrazino, carbamyl, phosphonic acid, phosphonato, or a straight, chained, branched, or cyclic alkyl, alkenyl, alkynyl, aryl heteroaryl, or heterocyclic group, which may be further substituted with any moieties such as hydrogen, halogen, hydroxyl, amino, amido, alkylamino, arylamino, alkoxy, aryloxy, nitro, acyl, alkenyl, alkynyl, cyano, sulfo, sulfato, mercapto, imino, sulfonyl, sulfenyl, sulfinyl, sulfamoyl, phosphonyl, phosphinyl, phosphoryl, phosphino, thioester, thioether, anhydride, oximno, hydrazino, carbamyl, phosphonic acid, phosphonato, or any other viable functional group; x (x′) and y (y′), independent of each other, are numbers from 1 to 3000. In certain embodiments, y (y′) is a number from 1 to 4 and x (x′) is a number from 1 to 100, from 1 to 20, from 1 to 10, or from 1 to 8.

Where the biguanide compound has at least four carbon-nitrogen double bonds (e.g. y≧2), hydrogen bonding results in the formation of a heterocyclic structure having the chemical formula of Formula IV:

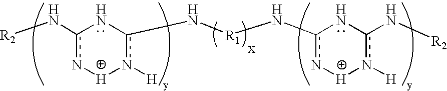

wherein R₁ comprises a straight, chained, branched, or cyclic alkyl group which may be further substituted with any moieties such as hydrogen, halogen, hydroxyl, amino, amido, alkylamino, arylamino, alkoxy, aryloxy, nitro, acyl, alkenyl, alkynyl, cyano, sulfo, sulfato, mercapto, imino, sulfonyl, sulfenyl, sulfinyl, sulfamoyl, phosphonyl, phosphinyl, phosphoryl, phosphino, thioester, thioether, anhydride, oximno, hydrazino, carbamyl, phosphonic acid, phosphonato, or any other viable functional group; R₂ and R₃, independent of one another, comprise a hydrogen, halogen, hydroxyl, amino, amido, alkylamino, arylamino, alkoxy, aryloxy, nitro, acyl, alkenyl alkynyl, cyano, sulfo, sulfato, mercapto, imino, sulfonyl, sulfenyl, sulfinyl, sulfamoyl, phosphonyl, phosphinyl, phosphoryl, phosphino, thioester, thioether, anhydride, oximno, hydrazino, carbamyl, phosphonic acid, phosphonato, or a straight chained, branched, or cyclic alkyl, alkenyl, alkynyl, aryl, heteroaryl, or heterocyclic group, which may be further substituted with any moieties such as hydrogen, halogen, hydroxyl, amino, amido, alkylamino, arylamino, alkoxy, aryloxy, nitro, acyl, alkenyl, alkynyl, cyano, sulfo, sulfato, mercapto, imino, sulfonyl, sulfenyl, sulfinyl, sulfamoyl, phosphonyl, phosphinyl, phosphoryl phosphino, thioester, thioether, anhydride, oximno, hydrazino, carbamyl, phosphonic acid, phosphonato, or any other viable functional group, x and y, independent of each other, are numbers from 1 to 3000.

The reaction between the biguanide compounds and base is believed to involve two different reaction mechanisms, largely depending upon the pH of the reaction conditions. It also is believed that under more basic conditions, the base reaction of the biguanide compound proceeds by the following mechanism to form a biguanide base.

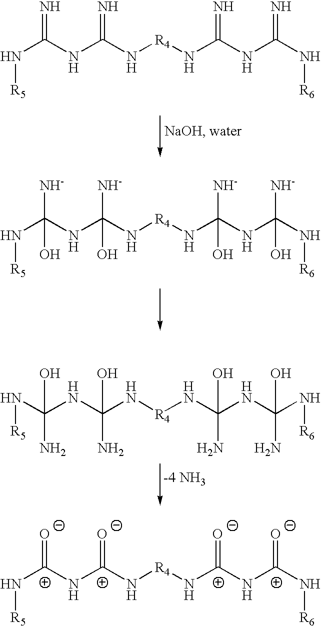

In the second reaction mechanism, it is believed that the biguanide compound (e.g., chlorhexidine) reacts with a dilute base to form a hydrated biguanide. Generally, hydrolysis of a biguanide would lead to the formation of ketone functionalities; however, elimination of the —NH₂ groups from the biguanide is either retarded or does not occur under mildly basic conditions, most likely due to strong intramolecular hydrogen bonding. Accordingly, it is believed that the soluble biguanide compound that undergoes hydrolysis has the above-described heterocyclic structure and forms a hydrated biguanide by the following mechanism.

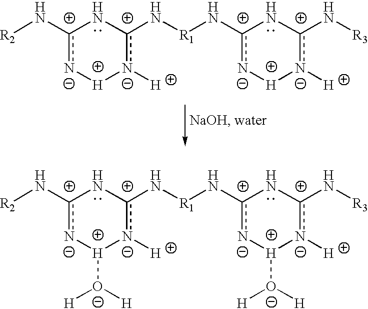

While not wishing to be bound by any theory, it is believed that the hydrate bonds formed in the foregoing reaction are hydrogen bonds rather than the covalent bonds which would be expected for most hydrates. Accordingly, the resulting material generally may be more temperature sensitive, and the temperatures the compound is exposed to during drying and after the reaction may be limited to the range of about 4° C. to about 40° C.

In embodiments wherein a salt of the biguanide compound is used as the initial reaction material, a reaction also may occur between the solubilizing agent and the anion (e.g., acetate). Such anions may be used to improve the solubility of biguanides such as chlorhexidine (e.g. chlorhexidine diacetate, chlorhexidine gluconate, or other soluble form of chlorhexidine).

Methods of Fluid Treatment and Devices Therefor

The composite materials described herein may be used in any suitable fluid treatment device. For example, in particular embodiments the device may comprise a packed particle bed of the composition. The device may include a housing for the packed particle bed having an inlet and an outlet with the packed particle bed disposed therebetween. In particular embodiments, the packed particle bed may further comprise a porous medium at the inlet and outlet to contain the composition within the device housing. Suitable housings, inlets, outlets, and porous media for such packed particle beds are well known to those of ordinary skill in the art.

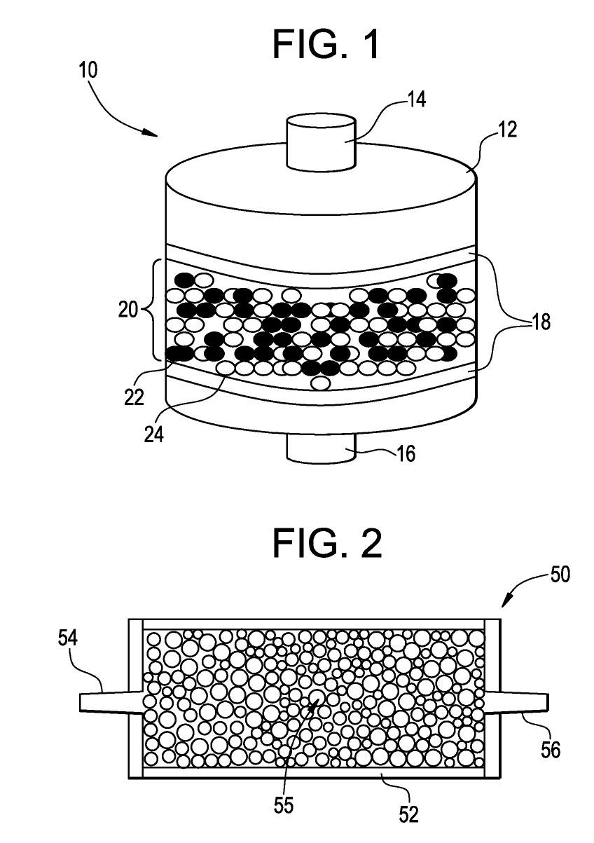

One embodiment of such a device is illustrated in FIG. 1. The device 10 includes a housing 12 having an inlet 14 and an outlet 16 for the flow of fluid therethrough. Disposed within the housing is a particle bed of the composite material 20 which includes carbon particles 22 and the antimicrobial material 24, such as chlorhexidine hydrate. The packed bed may be disposed between porous media 18 to contain the composite material 20 within the housing. The porous media may be a conventional filter media. such a porous polymeric material, for example which may be made of a polyethylene.

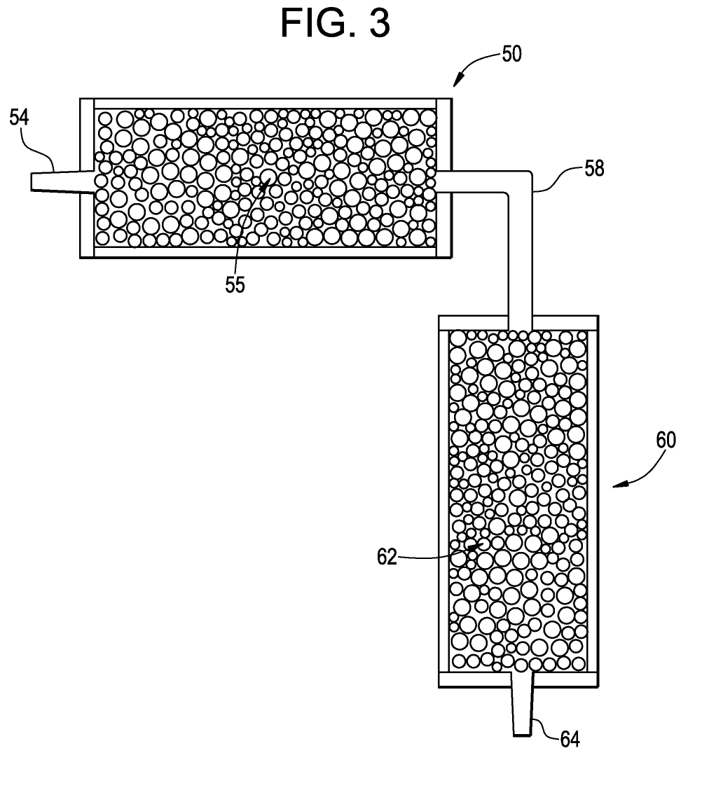

Another embodiment of a suitable device is illustrated in FIG. 2. The device 50 includes housing 52 having fluid inlet 54 and fluid outlet 56. An antimicrobial composite material 55, in the form of biguanide hydrate coated carbon particles, is provided as a fixed bed within the housing 52. In still another embodiment, which is illustrated in FIG. 3, the fluid outlet of device 55 is connected by conduit 58 to a secondary housing 60, which contains activated carbon particles 62 (without the biguanide hydrate). A fluid in need of treatment flows into fluid inlet 54, composite material 55 treats the fluid to deactivate any microorganisms in the fluid, then the fluid flows through the activated carbon 62 to remove any trace biguanide hydrate or other trace organic or metal contaminants from the fluid, and the treated fluid flows out of secondary fluid outlet 64.

This purification material or device may be used alone, or in combination with other materials and devices known in the art of fluid treatment. For instance, the purification material or device may be used in a process in series with a filtration device, for example as a pretreatment to remove larger-scale particulate matter and/or as a post treatment to filter out skeletal remains of inactivated microorganisms. As another example, the fluid may be treated using methods, materials, and systems known in the art to remove other organic or inorganic matter or solutes. Suitable filter media for pre-filtration are described for example in U.S. Pat. No. 6,187,192, No. 6,180,016, No. 6,957,743, No. 6,833,075; and No. 6,861,002; and in U.S. Patent Application Publication No. 2003/0173287 and No. 2004/0159605.

The antimicrobial composite materials may be particularly useful in those applications where the required reduction in the concentration of microbiological contaminants significantly exceeds the EPA standards for microbiological water purification devices. In one embodiment, the antimicrobial composite material comprises a biguanide hydrate, such as chlorhexidine hydrate, as described herein. In one method of using such an antimicrobial material, the microbiological contaminants are inactivated when the fluid is forced through the antimicrobial material by a difference in pressure on the influent and effluent sides or by a vacuum on the effluent side of the antimicrobial material.

The antimicrobial composite material may used as a purifier for drinking water. In another embodiment, the antimicrobial composite material may be used to purify water used in recreational settings, such as swimming pools, hot tubs, and spas. In such applications, the composite material may permit a reduction or elimination of chlorine usage, which is conventionally required to eliminate living microorganisms in such waters.

Because the antimicrobial composite material efficiently inactivates microorganisms in aqueous solutions, it may also have numerous applications in the pharmaceutical, medical, food, or beverage industries. It may, for example, be used for low-temperature sterilization, eliminating the need for techniques requiring elevated temperatures and pressures, such as pasteurization.

In another example, the antimicrobial composite materials may be used the purification of air or medical gases, such as in hospital or industrial areas requiring highly purified air having extremely low amounts of microorganisms, e.g., intensive care wards, operating rooms, clean rooms used for care of immunosuppressed patients, or industrial clean rooms for manufacturing electronic and semiconductor equipment.

The antimicrobial composite materials also may be used for residential air-purification. Such applications would be especially useful for individuals who suffer from heightened reactivity to air-borne microorganisms, such as fungi. In another embodiment, the antimicrobial composite material may be used to protect individuals from air-borne microorganisms in the event of a bioterrorist attack.

In one particular application, the antimicrobial composite materials may be incorporated into a device designed to eliminate pathogenic protozoa (e.g., of the genus Plasmodium and phylum Apicomplexa) that cause diseases such as malaria. Malaria is typically transmitted to humans through mosquitoes, which become infected with the protozoa from water reservoirs and lakes where the mosquitoes breed. The present antimicrobial composite materials may be used to assist in eliminating the protozoa from the breeding habitats of the mosquitoes, which could aid in eliminating malaria outbreaks.

Numerous other applications exist for which the present antimicrobial materials can be used. Representative examples include the treatment of water used in cooling systems, fermentation applications and cell culture, and inactivation of microorganisms in gases (e.g., anesthetics, carbon dioxide used in carbonated beverages, gases used to purge process equipment, etc.).

In each of these applications, the method of using the present antimicrobial materials is relatively simple: The fluid to be treated is brought into physical contact with the antimicrobial material of the composite. Typically, the fluid may be forced from one side of the composite material through pores in/among the antimicrobial material to the other side of the material due to gravity or a pressure drop across it. A conventional fluid pump, fan, or gravity feed can be used to drive the fluid contact.

The materials, devices, and methods described above will be further understood with reference to the following non-limiting examples.

### EXAMPLE 1

### Production of Chlorhexidine Hydrate

Commercially obtained chlorhexidine (C₂₂H₃₀N₁₀Cl₂), obtained commercially, was reacted with sodium hydroxide to form chlorhexidine hydrate. Approximately 100 g of a starting material chlorhexidine diacetate was dissolved in 1300 ml of warm deionized water at approximately 50° C. 6 M potassium hydroxide (KOH) was added drop-wise with stirring. A precipitate formed immediately and continued to form upon addition of base until the solution reached a pH of 11. The precipitate was filtered and washed six times with warm, 50° C., deionized water, and then dried in an oven at 60° C. to produce approximately 78 g of chlorhexidine hydrate.

The chlorhexidine hydrate has a theoretical formulation of C₂₂H₃₀N₁₀Cl₂.nH₂O. In multiple production runs, the chlorhexidine hydrate product was determined to have an actual degree of hydration (n) of about 1.4.

### EXAMPLE 2

### Production of Chlorhexidine Hydrate and Carbon Particle Mixture

Chlorhexidine hydrate prepared as described in Example 1. Activated carbon, derived from coconut shells, was obtained (Calgon Carbon #111270, Pentair Corp., Golden Valley, Minn.). The carbon particles were sieved, and the 40×80 mesh particles were well mixed with the chlorhexidine hydrate to form an antimicrobial composite material, which in this case was in the form of a particulate mixture.

The composite material was loaded as a fixed particle bed into a test apparatus, specifically into a device similar to that illustrated in FIG. 1. The device included an acrylic housing having an inlet and an outlet for the flow of fluid therethrough. The particle bed was 1.0 inch diameter×1.0 inch length (2.54 cm diameter×2.54 cm length) and was sandwiched between Porex™ (Fairburn, Georgia) polyethylene porous media, less than 25 micron thick.

Deionized water was inoculated with 4×10E+6 CFU E. Coli and flowed through the device at various flow rates using a positive pressure peristaltic pump. The bacterial recovery was determined by Aerobic Plate Count and is shown in Table 1.

TABLE 1
Reduction of Bacterial Growth in Water Using a
Chlorhexidine Hydrate and Carbon Composite

| Fraction Chlorhexidine | Fraction | Water Flow | Total E. Coli |
|---|---|---|---|
| Hydrate | Carbon | Rate | Aerobic Plate Count |
| (Wt %) | (Wt %) | (cc/min) | (CFU) |
| 0.00 | 100 | 17, 2, 71 | >1E+4 |
| 7.76 | 92.24 | 20, 55, 80 | >1E+4 |
| 13.89 | 86.11 | 23, 44, 69 | 0 |
| 23.05 | 76.95 | 16, 28, 64 | 0 |

Similar experiments were conducted with 50 and 80 mesh carbon and 8.40, 13.89, and 23.05% by weight of chlorhexidine hydrate. The mortality of E. Coli was greater than 10E+6 for mixtures having 13.89% by weight or greater chlorhexidine hydrate. Accordingly, it appears that the coarseness of the carbon is not critical to the antimicrobial effectiveness of the composite material.

### EXAMPLE 3

### Production of Chlorhexidine Hydrate Coated Carbon Particles

Chlorhexidine hydrate, prepared as in Example 1, was melted onto carbon particles by high shear mixing in a dough-like radial mixer at 110 to 125° C. The carbon particles included 40, 80 and 125 particle mesh size. The resulting mixture (i.e., chlorhexidine hydrate coated carbon particles) included approximately 24 to 60% by weight chlorhexidine hydrate.

A bed of the particle mixture was prepared in the device described in Example 2. Distilled water was made to flow through the particle bed under pumped or gravity flow conditions. The coated carbon particles allowed for continuous flow through the particle bed, even under gravity flow conditions, without the occurrence of channeling in the particle bed.

Approximately 5-32 ppm of chlorhexidine hydrate was detected in the effluent from the particle bed. However, when the effluent was subsequently treated with carbon alone, trace amounts of chlorhexidine hydrate were eliminated.

The coated carbon may particularly useful for water treatment because it does not require physical support, does not channel, and eliminates the occurrence of residual fine particulate in the effluent water.

Publications cited herein and the materials for which they are cited are specifically incorporated herein by reference. Modifications and variations of the methods and devices described herein will be obvious to those skilled in the art from the foregoing detailed description. Such modifications and variations are intended to come within the scope of the appended claims.

We claim:

1. An antimicrobial composite material comprising:
particles of carbon; and
an antimicrobial material comprising a compound having the formula 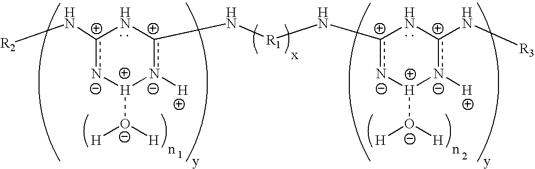
wherein R₁ comprises a straight chained, branched, or cyclic alkyl group;
wherein R₂ and R₃, independent of one another comprise a hydrogen, halogen hydroxyl, amino, amido, alkylamino, arylamino, alkoxy, aryloxy, nitro, acyl, alkenyl, alkynyl, cyano, sulfo, sulfato, mercapto, imino, sulfonyl, sulfenyl, sulfinyl, sulfamoyl, phosphonyl, phosphinyl, phosphoryl, phosphino, thioester, thioether, anhydride, oximno, hydrazino, carbamyl, phosphonic acid, phosphonato, or a straight, chained, branched, or cyclic alkyl, alkenyl, alkynyl, aryl, heteroaryl, or heterocyclic group;
wherein x is a number from 1 to 8 and y is a number from 1 to 4; and
wherein one or both of n₁ and n₂ is the number 1, wherein the composition has a degree of hydration greater than 0 and less than or equal to 2y.

2. The antimicrobial composite material of claim 1, which is in the form of a particulate mixture, wherein the antimicrobial material is in the form of particles dispersed among the particles of carbon.

3. The antimicrobial composite material of claim 2, wherein the particles of carbon are present in the particulate mixture in an amount from about 50% to about 85% by weight of the particulate mixture.

4. The antimicrobial composite material of claim 2, wherein the antimicrobial material is present in an amount from about 15% to about 50% by weight of the particulate mixture.

5. The antimicrobial composite material of claim 2, wherein the particulate mixture is in a porous compacted form having a volume average pore size between about 0.1 micron and about 5 microns.

6. The antimicrobial composite material of claim 1, wherein the antimicrobial material is in the form of a coating on the particles of carbon.

7. The antimicrobial composite material of claim 6, wherein the antimicrobial material is present in an amount from about 25% to about 60% by weight of the coated particles of carbon.

8. The antimicrobial composite material of claim 1, wherein the carbon particles are an activated carbon having a mesh size from about 40 to about 400 mesh.

9. The antimicrobial composite material of claim 1, wherein R₁ comprises a straight chained, branched, or cyclic alkyl group which is substituted with a moiety selected from the group consisting of hydrogen, halogen, hydroxyl, amino, amido, alkylamino, arylamino, alkoxy, aryloxy, nitro, acyl, alkenyl, alkynyl, cyano, sulfo, sulfato, mercapto, imino, sulfonyl, sulfenyl, sulfinyl, sulfamoyl, phosphonyl, phosphinyl, phosphoryl phosphino thioester, thioether, anhydride, oximno, hydrazino, carbamyl, phosphonic acid, and phosphonato.

10. The antimicrobial composite material of claim 1, wherein x is 6 and y is 1.

11. The antimicrobial composite material of claim 1, wherein R₁ is methylene.

12. The antimicrobial composite material of claim 1, wherein R₂ and R₃ are a halo-substituted phenyl.

13. The antimicrobial composite material of claim 1, wherein the antimicrobial material consists essentially of chlorhexidine hydrate which has about 4.0% to about 5.0% water by weight.

14. An antimicrobial composite material for treating fluids, comprising:
particles of an activated carbon; and
an antimicrobial material comprising a compound having the formula 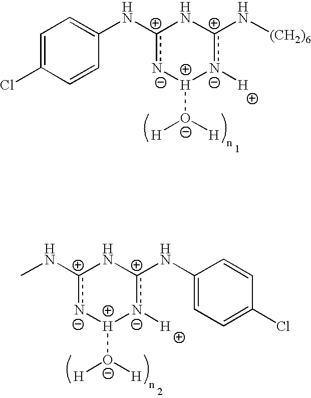
wherein one or both of n₁ and n₂ is the number 1, and
wherein the compound has a degree of hydration greater than 0 and less than 2.

15. The antimicrobial composite material of claim 14, wherein the compound has a degree of hydration between about 1.3 and about 1.6.

16. The antimicrobial composite material of claim 15, wherein the particles of activated carbon have a mesh size from about 40 to about 400 mesh.

17. The antimicrobial composite material of claim 16, wherein the particles of activated carbon have a mesh size from 40 to 80 mesh.

18. The antimicrobial composite material of claim 16, wherein the particles or activated carbon have a mesh size from 200 to 325 mesh.

19. The antimicrobial composite material of claim 14, which is in the form of a particulate mixture, wherein the antimicrobial material is in the form of particles dispersed among the particles of activated carbon.

20. The antimicrobial composite material of claim 19, wherein the particles of carbon are present in the particulate mixture in an amount from about 50% to about 85% by weight of the particulate mixture.

21. The antimicrobial composite material of claim 14, wherein the antimicrobial material is in the form of a coating on the particles of activated carbon.

22. The antimicrobial composite material of claim 15, wherein the antimicrobial material is present in an amount from about 15% to about 60% by weight of the antimicrobial composite material.

23. A device for inactivating microorganisms in a fluid comprising:
a housing having at least one fluid inlet and at least one fluid outlet; and
the antimicrobial composite material of claim 1 located within the housing between the at least one fluid inlet and the at least one fluid outlet.

24. The device of claim 23, further comprising at least one porous polymeric layer adjacent to the antimicrobial composite material.

25. The device of claim 23, wherein the antimicrobial composite material is sandwiched between two porous support layers.

26. The device of claim 23, further comprising a layer of particles of an activated carbon located between the antimicrobial composite material and the fluid outlet.

27. The device of claim 23, wherein the antimicrobial material consists essentially of chlorhexidine hydrate which has about 4.0% to about 5.0% water by weight.

28. The device of claim 27, wherein the antimicrobial composite material is in a porous compacted form having a volume average pore size between about 0.1 micron and about 5 microns.

29. A method for inactivating microorganisms in a fluid, comprising:
flowing a fluid in need of treatment through the antimicrobial composite material of claim 1 in a manner effective to inactivate at least one microorganism in the fluid.

30. A method for making an antimicrobial composite material, comprising the steps of
providing particles of a carbon; and
combining the particles of carbon with an antimicrobial material comprising a compound having the formula 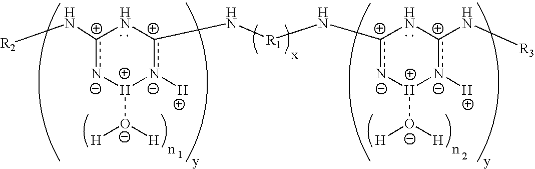
wherein R₁ comprises a straight chained, branched, or cyclic alkyl group;
wherein R₂ and R₃, independent of one another, comprise a hydrogen, halogen, hydroxyl, amino, amido, alkylamino, arylamino, alkoxy, aryloxy, nitro, acyl, alkenyl, alkynyl, cyano, sulfo, sulfato, mercapto, imino, sulfonyl, sulfenyl, sulfinyl, sulfamoyl, phosphonyl, phosphinyl, phosphoryl, phosphino, thioester, thioether, anhydride, oximno, hydrazino, carbamyl, phosphonic acid, phosphonato, or a straight, chained, branched, or cyclic alkyl, alkenyl, alkynyl, aryl, heteroaryl, or heterocyclic group;
wherein x is a number from 1 to 8 and y is a number from 1 to 4; and
wherein one or both of n₁ and n₂ is the number 1, wherein the composition has a degree of hydration greater than 0 and less than or equal to 2y.

31. The method of claim 30, wherein the antimicrobial material is provided in the form of particles which then are mixed with the particles of carbon.

32. The method of claim 30 wherein the antimicrobial material is heated above its melting temperature, coated onto the particles of carbon, and then cooled to below its melting temperature.

33. The method of claim 30, wherein the antimicrobial material comprises a compound having the formula 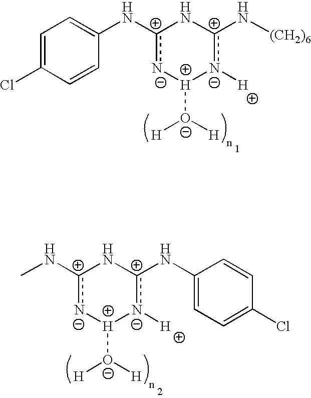
wherein one or both of n₁ and n₂ is the number 1, and
wherein the compound has a degree of hydration greater than 0 and less than 2.

34. The method of claim 33, wherein the compound has a degree of hydration between about 1.3 and about 1.6.

35. An antimicrobial composite material comprising:
particles of an inert substrate material, the particles having a volume average volume average size between about 100 microns and about 5 mm; and
an antimicrobial material coated onto the particles of an inert substrate material, wherein the antimicrobial material comprises a compound having the formula 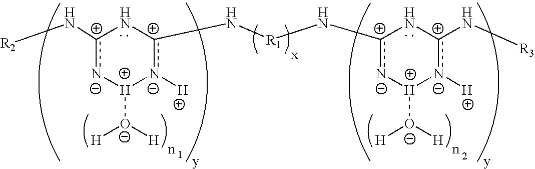
wherein R₁ comprises a straight chained, branched, or cyclic alkyl group;
wherein R₂ and R₃, independent of one another, comprise a hydrogen, halogen, hydroxyl, amino, amido, alkylamino, arylamino, alkoxy, aryloxy, nitro, acyl, alkenyl, alkynyl, cyano, sulfo, sulfato, mercapto, imino, sulfonyl, sulfenyl, sulfinyl, sulfamoyl, phosphonyl, phosphinyl, phosphoryl, phosphino, thioester, thioether, anhydride, oximno, hydrazino, carbamyl, phosphonic acid, phosphonato, or a straight, chained, branched, or cyclic alkyl, alkenyl, alkynyl, aryl, heteroaryl, or heterocyclic group;
wherein x is a number from 1 to 8 and y is a number from 1 to 4; and
wherein one or both of n₁ and n₂ is the number 1, wherein the composition has a degree of hydration greater than 0 and less than or equal to 2y.

36. The antimicrobial composite material of claim 35, wherein the antimicrobial material comprises a compound having the formula 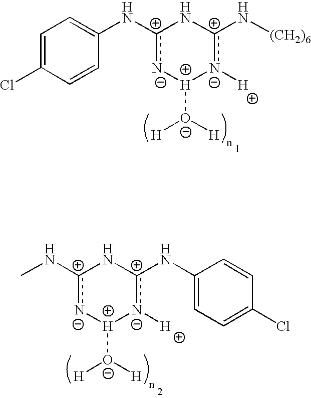
wherein one or both of n₁ and n₂ is the number 1, and
wherein the compound has a degree of hydration greater than 0 and less than 2.

37. The antimicrobial composite material of claim 35, wherein the compound has a degree of hydration between about 1.3 and about 1.6.

38. The antimicrobial composite material of claim 37, wherein the inert substrate material comprises a ceramic, a noble metal, a polytetrafluoroethylene, or a glass.
